(19) United States
(12) Patent Application Publication
(10) Pub. No.: US 2015/0126913 A1
(43) Pub. Date: May 7, 2015

(54) METHOD AND SYSTEM FOR SKIN TREATMENT

(71) Applicant: KONINKLIJKE PHILIPS N.V., Eindhoven (NL)

(72) Inventors: Martin Jurna, Eindhoven (NL); Jonathan Alambra Palero, Eindhoven (NL); Margaret Ruth Horton, Eindhoven (NL)

(21) Appl. No.: 14/394,747

(22) PCT Filed: Apr. 12, 2013
(86) PCT No.: PCT/IB2013/052912

§ 371 Date: Oct. 16, 2014

Related U.S. Application Data
(60) Provisional application No. 61/624,521, filed on Apr. 16, 2012.

(51) Int. Cl. A61B 18/20; A61M 35/00; A61H 9/00; A61B 18/12

(52) U.S. Cl. CPC A61B 18/203; A61B 18/12; A61M 35/00; A61H 9/0057; A61B 2018/00452
USPC 601/9; 606/41; 606/34; 606/2; 604/20

(57) ABSTRACT

A method of skin tissue (1) treatment is provided which comprises the steps of: determining a treatment zone (9) within the skin tissue below the skin surface (3); modifying an electrical conductance property of at least two first skin tissue portions (11) present on opposite sides of the treatment zone with respect to a direction parallel to the skin surface; and providing radio frequency (RF) energy to the treatment zone via said first skin tissue portions. The step of modifying said first skin tissue portions comprises decreasing electrical impedance for the radiofrequency energy, in particular increasing electrical conductance, of said first skin tissue portions relative to a second skin tissue portion present between said first skin tissue portions; and such that said first skin tissue portions extend into the skin tissue substantially from the skin surface to treatment zone. A system for skin tissue (1) treatment is also provided.

### FIELD OF THE INVENTION

The present disclosure relates to treatment of mammalian tissue, in particular human skin and subdermal tissue, more in particular it relates to heat treatment by radiofrequency energy for skin tightening and/or skin rejuvenation.

### BACKGROUND OF THE INVENTION

It is known that human skin may be rejuvenated if the skin is intently heated to a temperature that is significantly above normal body temperature so as to induce intently small-scale tissue injury and/or minor damage, collagen denaturation and/or coagulation, tissue ablation and/or necrosis. This urges the body to respond by restoring the damaged tissue, which results in the desired tightened and rejuvenated skin.

For successful treatment, the target tissue zone or treatment zone should be properly addressed and other tissue should be spared. The treatment zone for skin rejuvenation is generally in the cutis (epidermis and dermis) and subcutis. For localized heating, U.S. Pat. No. 7,955,262 discloses a system and method for treating skin, to obtain the aesthetic effect of skin rejuvenation by using radiofrequency (RF) energy to heat the tissue. The RF treatment is preceded by first directing acoustic energy at ultrasound wavelengths to the skin surface. This provides a first heating of the tissue at the focal volumes of the ultrasound energy. RF energy is subsequently applied to the skin and the RF current is guided into the focal volumes preheated by the ultrasound energy. According to U.S. Pat. No. 7,955,262 it is believed that this guiding effect is based on the temperature dependence of RF conductivity on the tissue temperature and to prevent damaging of tissue surrounding focal volumes to be heated and the remainder of the treatment zone, the treated zone should preferably be cooled prior to application of the energy sources.

However, ultrasound tends to interact with biological tissues not only thermally but also mechanically (even at low pressure levels), specifically by the generation of cavitation bubbles, which is deemed undesirable and unsafe for biological tissues. Furthermore, scattering of deep-penetrating focused or unfocused ultrasound energy can result in hotspots inside tissues which is a serious safety concern. Control of the amount, location and temperature of the pre-heating and therewith of the RF heating, and thus of the treatment as a whole, is therefore inadequate or at least very complicated.

### SUMMARY OF THE INVENTION

In order to improve skin tissue therapy the method and system defined in the appended claims are provided herewith.

The method of skin tissue treatment, in particular being a method for cosmetic skin tightening and skin rejuvenation, comprises the steps of: determining a treatment zone within the skin tissue below the skin surface; modifying an electrical conductance property of at least two first skin tissue portions present on opposite sides of the treatment zone with respect to a direction parallel to the skin surface and providing radiofrequency energy to the treatment zone to heat the treatment zone. The step of the modifying said first skin tissue portions comprises decreasing electrical impedance for the radiofrequency energy, in particular increasing electrical conductance, of said first skin tissue portions relative to a second skin tissue portion present between said first skin tissue portions and such that said first skin tissue portions with a decreased electrical impedance extend into the skin tissue substantially from the skin surface to the treatment zone.

The first skin tissue portions, hereafter also called “low-impedance portions”, provide channels into the skin to the treatment zone within the skin having reduced losses for radiofrequency (RF) energy compared to skin tissue surrounding the first skin tissue portions that is not modified. Due to the effectively increased conductance with respect to the surrounding tissue, the RF energy is preferentially guided to the treatment zone by said low-impedance portions and dissipation of the RF energy in the first skin tissue portions is reduced compared to skin tissue that is not modified. This improves effective penetration depth of the RF energy and it improves accuracy of the application of the RF energy, as well as increases the usable RF energy in the treatment zone. Due to the low-impedance portions extending substantially from the skin surface to the treatment zone or, depending on the point of view, from the treatment zone up to the skin surface, electrical contact resistance between the source of RF energy and the low-impedance portion is decreased, improving incoupling of the RF energy into the channels improving effectiveness of the method.

The low-impedance skin tissue portions may be, but need not be, substantially straight.

In another aspect, a system for skin tissue treatment, in particular for performing one or more aspects of the method generally outlined above is provided herewith. The system comprises a radio frequency source for providing radio frequency energy to the treatment zone of the skin tissue to heat said treatment zone, comprising a radiofrequency (RF) energy source with one or more radiofrequency electrodes. The system further comprises a modifier configured to modify an electrical conductance property of at least two first skin tissue portions for guiding the radio frequency energy from the one or more radiofrequency electrodes through said first skin tissue portions to the treatment zone. The modifier for modifying an electrical conductance property of at least two first skin tissue portions for guiding the radiofrequency energy from the one or more radiofrequency electrodes through said first skin tissue portions to the treatment zone for guiding the radiofrequency energy from the one or more radiofrequency electrodes through said first skin tissue portions to the treatment zone. The modifier is configured to decrease the electrical impedance for the radiofrequency energy, in particular increase electrical conductance, of at least two first skin tissue portions relative to a second skin tissue portion present between said first skin tissue portions, wherein the first skin tissue portions are present on opposite sides of the treatment zone with respect to a direction parallel to the skin surface and extend into the skin tissue substantially from the skin surface toward the treatment zone. Advantageously, the device for locally increasing electrical conductance is configured to heat skin tissue and/or to provide a fluid-filled cavity in the skin tissue.

The method of claim 2 and likewise the system of claim 9 facilitates guiding of the RF energy into the treatment zone since the path of least resistance for the RF energy extends between the portions of the first skin tissue portions that are closest to each other. In case of substantially straight channels extending toward each other within the skin tissue, such low-resistance path extends between the respective tips of the channels. In case of one or more curved channels and/or having a varying width along the axis of extension, a close separation may also be provided at one or more other portions than the tips along the length of one or both channels.

The method of claim 3 employs the positive correlation of heating of skin tissue tending to increase its electrical conductivity. Localised heating of skin tissue may be realised by various reliable techniques. A further benefit of the method is that heating of the skin tissue may be non-invasively and transient, leaving no lasting effects. In another embodiment, the heating may cause thermal injury, which may be beneficial for inducing skin rejuvenation as well.

The system of claim 10 facilitates accurate control over heating of one or more skin tissue portions to decrease the electrical impedance thereof. Laser beams may be reliably directed, focused, power-controlled, intensity controlled and/or switched etc. with well-proven technology. Numerous Lasers emitting different wavelengths, powers etc. with associated different effects are commercially available. In particular infrared (IR) radiation wavelengths in the infrared spectrum between about 1-10 micrometers show useful combinations of penetration depths and absorption into mammalian, in particular human, skin tissue. Combinations of plural wavelengths may be used to provide particular electrical impedance variations in the skin tissue, e.g. with respect to size and/or location within the skin tissue.

The method of claim 4 and likewise the system of claim 11 benefits from the effect that ablating skin tissue provides a layer of heated skin tissue adjacent the ablated zone which has a relatively high electrical conductivity, whereas the burnt tissue or ablated zone has a relatively very low electrical conductivity compared to unaffected tissue. Hence, the low-impedance zone is well-defined and RF energy may be directed away from the burnt or ablated zone and guided more effectively into the surrounding tissue.

The method of claim 5 and likewise the system of claim 12 facilitates providing a large difference in impedance between the fluid-filled cavities and the surrounding tissue. Said cavity(s) may be formed by the application of the fluid itself by a suitable dispenser, e.g. due to an injection with a physical applicator such as a hollow needle, a syringe and/or by direct dispensing the fluid in the form of a forceful fluid jet.

In an aspect, a cavity may be made in the skin tissue by burning and/or ablating tissue.

The fluid may be provided from an external source, e.g. water, saline, etc. and/or comprise a body fluid of the treated subject, e.g. interstitial fluid, lymph and/or blood. The latter method may efficiently be combined with burning or ablating a tissue portion to provide an open cavity that is at least partly filled with a body fluid, where the conductance of heated skin tissue adjacent the cavity is exploited concurrent with and/or directly subsequent to the burning and/or ablation step and during the filling of the cavity with body fluid that takes over the role of the high-conductivity portion as the tissue cools.

Filling such cavity with one or more body fluids may be assisted by applying a pressure difference across one or more of said cavities between the skin tissue and the surrounding atmosphere, e.g. by applying a negative pressure or suction to the cavity(s) and/or applying positive pressure to tissue adjacent the cavity(s).

The method of claim 7 improves incoupling of the RF energy into the low-impedance skin tissue portion by reduction of the physical (and electromagnetic) path length between the electrodes and said skin tissue portion(s).

The system of claim 14 facilitates providing close contact between the RF electrodes and the low-impedance skin tissue portion, at least a portion of the first and second patterns may be substantially identical.

Closest contact is direct physical contact with said skin tissue portion(s). The electrical contact may be improved by use of impedance matching fluids, e.g. conductive creams and/or gels. In an advantageous embodiment, plural RF electrodes are used, each in close contact with another low-impedance skin tissue portion. The electrodes may wholly or partly surround and/or overlap the positions of the skin surface in which the modifier interacts with the skin tissue.

### BRIEF DESCRIPTION OF THE DRAWINGS

In the drawings:

FIG. 1 indicates RF heating of skin tissue without providing low-impedance portions;

FIGS. 2A and 2B indicate two embodiments of RF heating of skin tissue according to the present disclosure;

FIGS. 3A and 3B indicate electrical equivalent schemes for RF heating of skin tissue according to the present disclosure;

FIGS. 4A-4R illustrate the results of simulations of RF heating of skin tissue according to the present disclosure with different parameters and compared to prior art;

FIG. 5 illustrates a system for RF treatment of skin tissue according to the present disclosure;

FIG. 6 illustrates a detail of an embodiment of a system for RF treatment of skin tissue according to the present disclosure;

FIG. 7 illustrates a detail of another embodiment of a system for RF treatment of skin tissue according to the present disclosure;

FIG. 8 illustrates a method of providing a fluid-filled cavity in skin tissue;

FIG. 9 illustrates a further detail of another embodiment of a system for RF treatment of skin tissue according to the present disclosure;

FIGS. 10A-10E indicate different suitable geometries for RF electrodes.

### DETAILED DESCRIPTION OF EMBODIMENTS

It is noted that in the drawings, like features may be identified with like reference signs. It is further noted that the drawings are schematic, not necessarily to scale and that details that are not required for understanding the present invention may have been omitted. The terms “upward”, “downward”, “below”, “above”, and the like relate to the embodiments as oriented in the drawings. Further, elements that are at least substantially identical or that perform an at least substantially identical function are denoted by the same numeral.

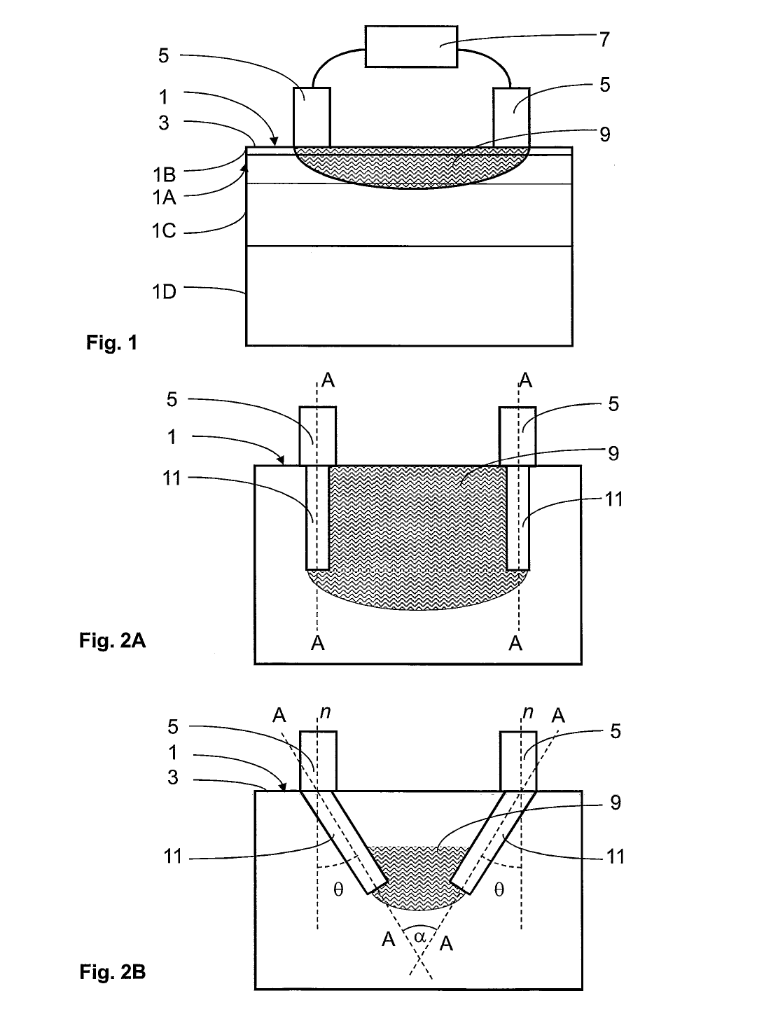

FIG. 1 schematically indicates RF treatment of skin tissue 1 having a skin surface 3 and tissue layers epidermis 1A (including the stratum corneum 1B), dermis 1C and subcutis 1D. The treatment uses a treatment system comprising RF electrodes 5 connected to an RF source 7. The electrodes 5 are placed in contact with the skin surface 3 at some distance from each other. By applying an RF signal to the electrodes 5, an RF current will flow through the skin between the two electrodes 5 and RF energy will be provided to the skin tissue 1 in a treatment zone 9. As a result, the treatment zone 9 between the two electrodes is heated. When in such manner tissue in the dermis layer 1C is heated to temperatures between 60° C. and 80° C., the collagen in the dermis will contract. The resulting effect is tightening of the skin, wrinkle reduction, and the reduction of fine lines and skin sagging. The resulting synthesis of new collagen can lead also to skin rejuvenation. The RF energy will be distributed along the path of least resistance between the RF electrodes. Hence, the skin tissue zone 9 that can be treated this way extends little depth into the skin, and the penetration depth into the skin tissue is difficult to control or select, if possible at all.

FIGS. 2A and 2B indicate embodiments of significant improvements. Different from FIG. 1, in FIGS. 2A, and 2B two first skin tissue portions 11 are present on opposite sides of the treatment zone 9 with respect to a direction parallel to the skin surface 3 which have decreased electrical impedance for the radio frequency energy relative to the skin tissue between the first skin tissue portions 11. The shown first skin tissue portions 11 have a substantially straight elongated shape with a longitudinal axis A, e.g. columns or plate-like shapes with respect to a direction out of the Figure-plane, and they extend into the skin tissue 1 from the skin surface 3 to the treatment zone 9. In FIG. 2A the longitudinal axes A of the shown pair of low-impedance first skin tissue portions 9 extend substantially parallel to each other into the skin 1, here being substantially perpendicular to the skin surface 3. In FIG. 2B the low-impedance first skin tissue portions 9 extend obliquely into the skin tissue 1 at an angle θ with respect to the normal n to the skin surface 3 so that the longitudinal axes A of the elongated skin tissue portions of the pair converge toward each other at an angle of convergence α in a direction from the skin surface 3 toward the treatment zone 9.

When an RF signal is applied to the electrode 5, the RF energy will flow through the low-impedance portions 11 and through the skin tissue present between them, which will be heated thereby. Due to the reduced impedance and in accordance with Ohms law, the RF energy will preferentially flow through the low-inductance portions 11 rather than through skin portions with higher impedance. Hence, the RF energy will penetrate relatively deep into the skin tissue 1, so that a treatment zone 9 extending deep into the skin 1 or localized deep within the skin 1 may be treated effectively and controllably by appropriately forming the first skin tissue portions 11.

In the embodiment of FIG. 2B, due to the converging first skin tissue portions 11, the path with least impedance is formed between the end points of the low-impedance skin tissue portions 11. Hence, the RF energy will predominantly flow through the skin tissue 1 at that depth, forming a treatment zone 9 that is localised deep within the skin tissue 1.

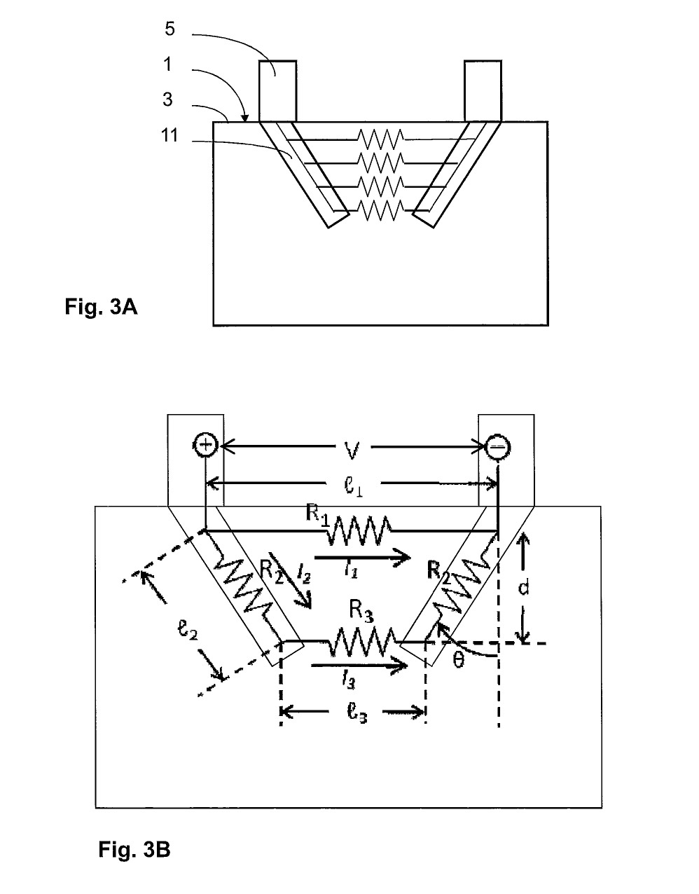

Without wishing to be bound by any specific theory and for general understanding of the working principles of the presently provided method, consider the following with reference to FIGS. 2B, 3A and 3B. The RF energy may be treated as an electrical signal travelling through an electrically conductive network, providing a plurality of conductive paths in parallel, each path i having its own resistance Rᵢ, see FIG. 3A. A simplified dual-layer configuration is shown in FIG. 3B for further exemplary purposes.

The RF heating mainly occurs at the location where the tissue has the highest current flow and resistance. Specifically, the locally produced heat Qᵢ equals the locally deposited power and is proportional to the square of the local current I multiplied by the local electric resistance R (series circuit) as

Q∝I²R. (Eq. 1)

Since the current is dependent on the electric potential V and resistance as

I=V/R, (Eq. 2)

the heat produced can be expressed as

Q∝V²/R. (Eq. 3)

Note that this shows that one can indeed guide the electric current flow and consequently localize heating by modifying the tissue's resistance. In the present disclosure the tissue is locally heated and/or fluid-filled to guide the electric current into deep areas of the skin, allowing deeper penetration of RF energy into the skin.

In FIG. 3B, the currents I₁ and I₂, respectively, are determined by the resistors R₁ of a skin surface layer, and, respectively by R₂ of the low-impedance first skin tissue portions 11 on either side of the treatment zone 9 and R₃ of the treatment zone 9. The resistance Rᵢ and local temperature change ΔTᵢ of each path section i is determined by its length lᵢ, its specific conductance σᵢ and its cross sectional area Aᵢ. The local temperature change ΔT₃ at R₃ (with length l₃) due to the RF current-produced heat Q₃ follows the relation

ΔT₃∝Q₃/l₃A₃. (Eq. 4)

This can be rewritten, using Eq. (1), as

ΔT₃∝I₃²R₃/l₃A₃. (Eq. 5)

Since I₃=V/(R₂+R₃+R₂) and Rᵢ=lᵢ/(σᵢAᵢ), the depth-dependent temperature change can be expressed as

ΔT₃(d)=σ₃/{(2σ₃d/σ₂ cos θ)−(2d tan θ)+l₁}². (Eq. 6)

Human skin tissue is generally electrically conductive. For an RF frequency of 1 MHz, the electrical conductivity C of different types of human tissue is given in Table 1, in units of S m⁻¹ (from: Sadick and Makino in: Lasers in Surgery and Medicine 34:91-97 (2004)).

TABLE 1
Electrical conductivity C of different types of human
tissue for RF radiation at a frequency of 1 MHz

| Tissue type | C [S m⁻¹] |
|---|---|
| Blood | 0.7 |
| Bone | 0.02 |
| Fat | 0.03 |
| Dry skin surface | 0.03 |
| Wet skin surface | 0.25 |

Further, the thermal coefficient of the skin conductance is approximated to be 2% ° C.⁻¹ (Sadick and Markino, op.cit.), so that raising the tissue temperature lowers the electrical resistance of the tissue.

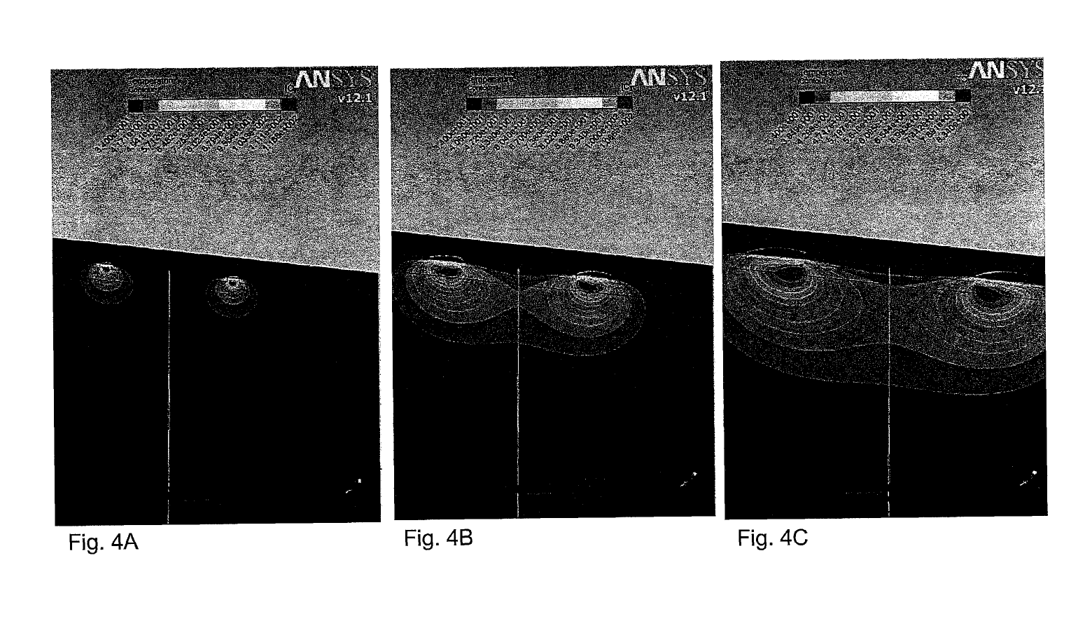

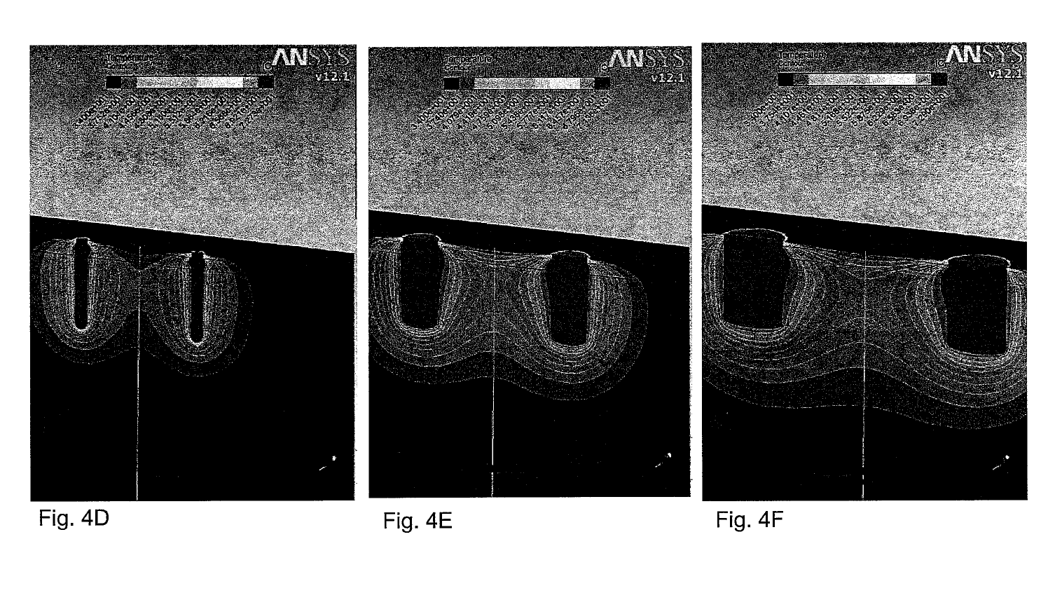

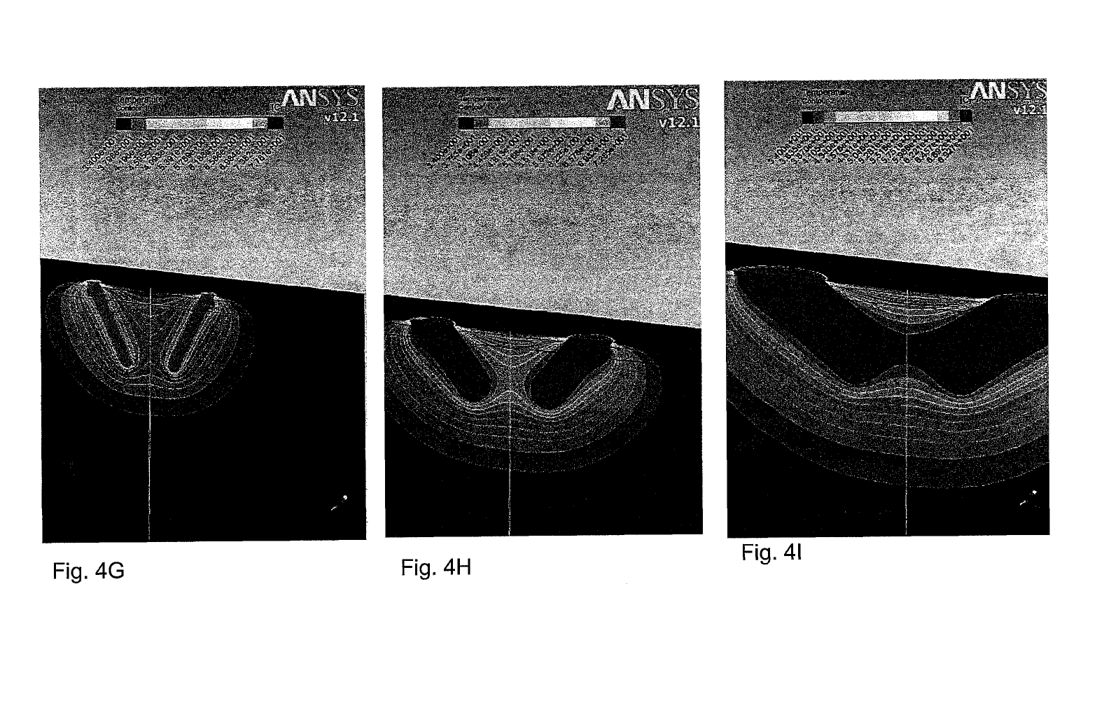

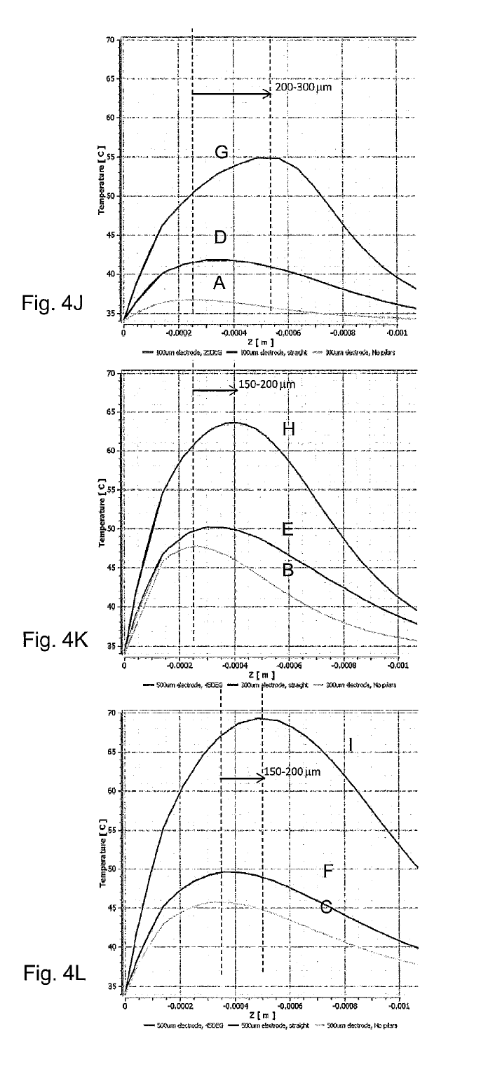

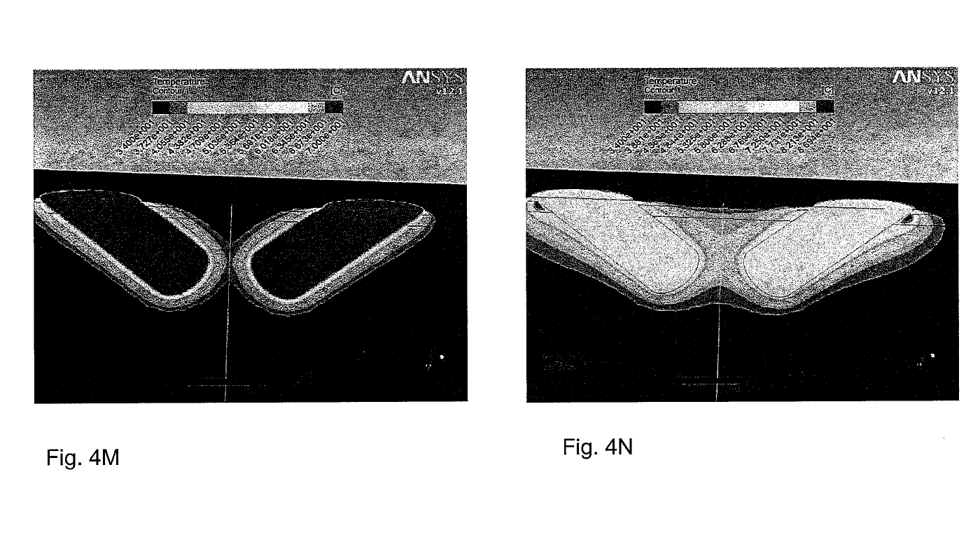

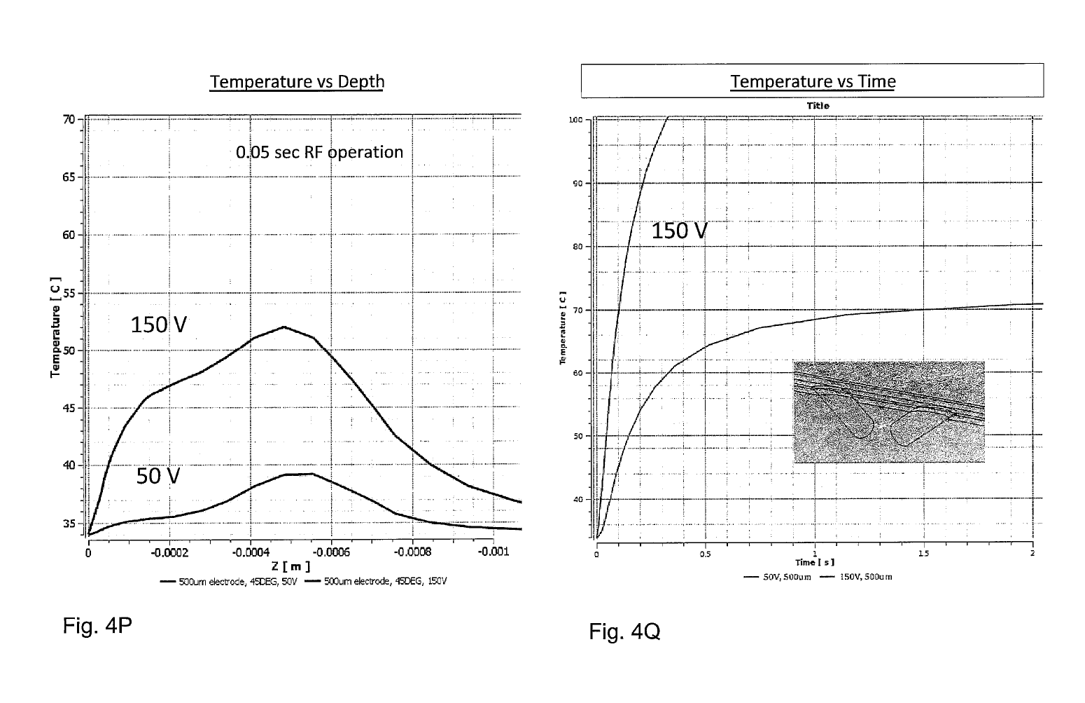

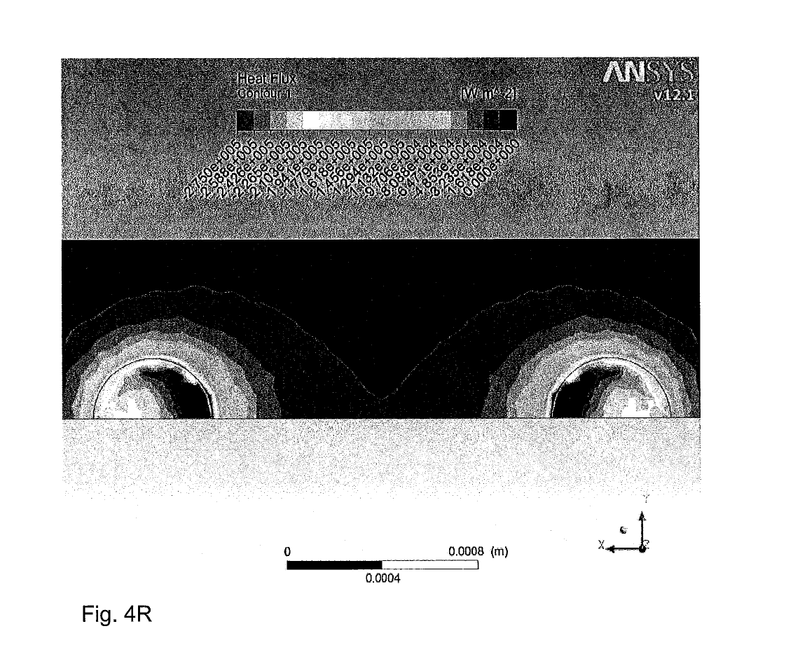

The results of detailed numerical simulations of different parameter configurations are shown in FIGS. 4A-4R, in which further effects like dielectric heating have been taken into account as well. FIGS. 4A-4C show simulations of the situation of FIG. 1, FIGS. 4D-4F generally correspond to the situation of FIG. 2A and FIGS. 4G-4I generally correspond to the situation of FIGS. 2B. FIGS. 4J-4L show a comparison of the results 4A, 4D, 4G/4B, 4E, 4H/4C, 4F, 4I, respectively. FIGS. 4M-4N show the situation of FIGS. 4G-4I with different operating parameters and FIGS. 4P and 4Q show a comparison of the results of FIGS. 4M-4N. FIG. 4R is a top view of the situation of FIG. 4M.

In the simulations the skin surface temperature is maintained at normal human skin temperature of 34° C. by suitable cooling, and the first skin tissue portions 11 are prepared by heating columns of skin tissue to 70° C. This temperature was maintained constant as well. The first skin tissue portions 11 are generally columnar with a length along the longitudinal axis A of about 1 mm, and extend at an angle θ into the skin. The RF electrodes have identical sizes as the first skin tissue portions and both are at a separation at the skin surface 3 of about 1 mm or 1.4 mm in the case of FIGS. 4C, 4F and 4I. The RF frequency was 1 MHz, with an arbitrarily determined value for the signal amplitude of 50 V root mean square (rms), with 150 V rms used in FIG. 4N. 50V rms corresponds to an amount of dissipated heat of about 0.1 W after 1 second of RF operation. It is noted that for some treatments the RF frequency of choice may be different. In the simulations it was further assumed that the stratum corneum was well hydrated.

Taking the values of Table 1, and assuming substantially constant resistance at the used RF frequency yields

σ₃=σ(T=35° C.)≈0.25

σ₂=σ(T=70° C.)≈0.50

Further, the distance between the RF electrodes on the skin surface 3 l₁ is taken to be 5 mm and equal to the local separation of the first skin tissue portions 11.

In FIGS. 4A-4C, no preheated first skin tissue portions are prepared and all effects are due to an RF field from RF electrodes placed on the skin (cf. FIG. 1). The RF electrodes are simulated to provide a circular contact portion to the skin surface of 100 micrometer diameter (FIG. 4A), 300 micrometer diameter (FIG. 4B), or 500 micrometer diameter (FIG. 4C). In FIGS. 4D-4F preheated first skin tissue portions are prepared which extend into the skin tissue substantially perpendicular to the skin surface with diameters 100, 300, and 500 micrometer, respectively, like in FIGS. 4A-4C. In FIGS. 4G-4I preheated first skin tissue portions are prepared which extend into the skin tissue at an oblique angle of 25° (FIG. 4G) or 45° (FIGS. 4H-4I), with diameters 100, 300, and 500 micrometer, respectively, like in FIGS. 4A-4C. In FIGS. 4G-4I, the oblique angles θ of the preheated first skin tissue portions 11 with respect to a plane comprising the pair of first skin tissue portions 11 under consideration is used as a parameter: θ=25° (FIG. 4G) and θ=45° (FIGS. 4H, 4I). Here, the angles θ are substantially identical for both columns of heated skin tissue, but this is not required and different angles may be provided including having one first skin portion extending substantially perpendicular to the skin surface and one or more first skin portions extending towards the perpendicular first skin portion at an acute angle to the skin surface. One first skin portion may be surrounded by plural first skin tissue portions and be used as a common pole for connection to an RF electrode of one polarity with respect to the surrounding portions being connected to RF electrodes at the opposite polarity.

FIGS. 4A-4I, and 4M-4N show isotherms separated by equal temperature intervals over different amounts of degrees heating over the initial temperature. In FIG. 4A the scale ranges from 3.40 to 11.88 degrees heating, in FIG. 4B the scale ranges from 3.40 to 10.66 degrees heating, in FIG. 4C the scale ranges from 3.40-8.34 degrees heating, in FIG. 4D the scale ranges from 3.40 to 7.273 degrees heating, in FIG. 4E the scale ranges from 3.40-7.136 degrees heating, in FIG. 4F the scale ranges from 3.40-7.29 degrees heating, in FIG. 4G the scale ranges from 3.40-7.81 degrees heating, in FIG. 4H the scale ranges from 3.40-7.285 degrees heating, in FIG. 4I the scale ranges from 3.40-6.927 degrees heating, in FIG. 4M the scale ranges from 3.40-7.00 degrees heating, in FIG. 4N the scale ranges from 3.40-8.694 degrees heating. FIG. 4R similarly shows iso-heat flux contours equally divided in a range of 0 to −2.750×10⁵ W/m².

FIG. 4J shows the depth-dependency of the tissue temperature change of the skin tissue in a plane central between the electrodes 5 into the skin for the simulation results of FIGS. 4A, 4D, 4G, as indicated with the respective letters in FIG. 4J. Similarly FIG. 4K relates to FIGS. 4B, 4E and 4H, and FIG. 4L relates to FIGS. 4C, 4F, and 4I.

FIGS. 4A-4L clearly show that, as expected and indicated before, localised skin tissue portions having reduced impedance, in particular pre-heated columns of tissue at 70° C., can be used to guide RF energy and heat sub-surface tissue between the columns and for oblique columns between the column ends. The penetration depth of the RF heating into the skin is significantly increased. This sub-surface RF heating (FIGS. 4D-4I) allows treatment of a larger tissue volume than the conventional RF electrode-only configuration (FIGS. 4A-4C). The penetration depth and localisation are controllable by selecting the oblique angle θ, and consequently of the angle of convergence α. Other control parameters are the diameter of the first skin tissue portions 11 and the RF power, e.g. determined by the rms value of the RF signal. E.g. FIGS. 4M-4Q indicate that increasing the rms value of the RF energy threefold, but keeping all other parameters equal, the peak temperature in the skin tissue after 0.05 seconds RF energy deposition has in creased from about 4° C. at 50 V rms to about 18° C. at 150 V rms (FIG. 4P), and the temperature between the first skin tissue portions at a depth of about 500 micrometer below the skin surface continues to rise significantly instead of levelling off (FIG. 4Q; the location considered is indicated in the inset).

FIG. 4R indicates the spatial extent of the heating of FIG. 4M on the skin surface, showing that the temperature indeed increases predominantly in the skin tissue located between the columns 11. Similar to FIGS. 4A-4I, and 4M-4N, FIG. 4R shows iso-heat flux contours equally divided in a range of 0 to −2.750×10⁵ W/m².

It is noted that application of RF energy to skin also the temperature of the low-inductance skin tissue portions will increase. This may be suitably employed for skin treatment as well.

For some treatments the RF frequency of choice may differ from 1 MHz.

Larger diameters of the first skin portions are found to guide the RF energy better than smaller diameters. Providing plural low-impedance portions adjacent each others to form an array (e.g. in a generally linear direction) improves guiding of the RF heating between the electrodes. Without such array, the RF energy dissipation is distributed over a larger volume of tissue.

In the simulated configurations, the heat flux that is created by the RF electrodes placed on the skin surface on top of the first skin tissue columns can easily be removed by surface cooling, if so desired, e.g. to better localise the treatment zone within the skin below the skin surface.

It is expected that instead of using heated skin tissue portions, ablated portions filled with high conductance liquid are used, the result will be an even better guiding (see also below).

Similar to the 3 dimensional geometry presented above, it has been calculated that for a substantially 2-dimensional geometry, e.g. plate-like first skin tissue portions extending adjacent each other at constant separation, for an angle θ=ca 30° (α=ca. 120°), a substantially homogenous temperature increase profile may be found, whereas for an angle θ>ca 30° (or conversely α<ca. 120°), the heating occurs predominantly deep within the skin with a decreasing gradient towards the skin surface 3, as shown for the 3-dimensional case.

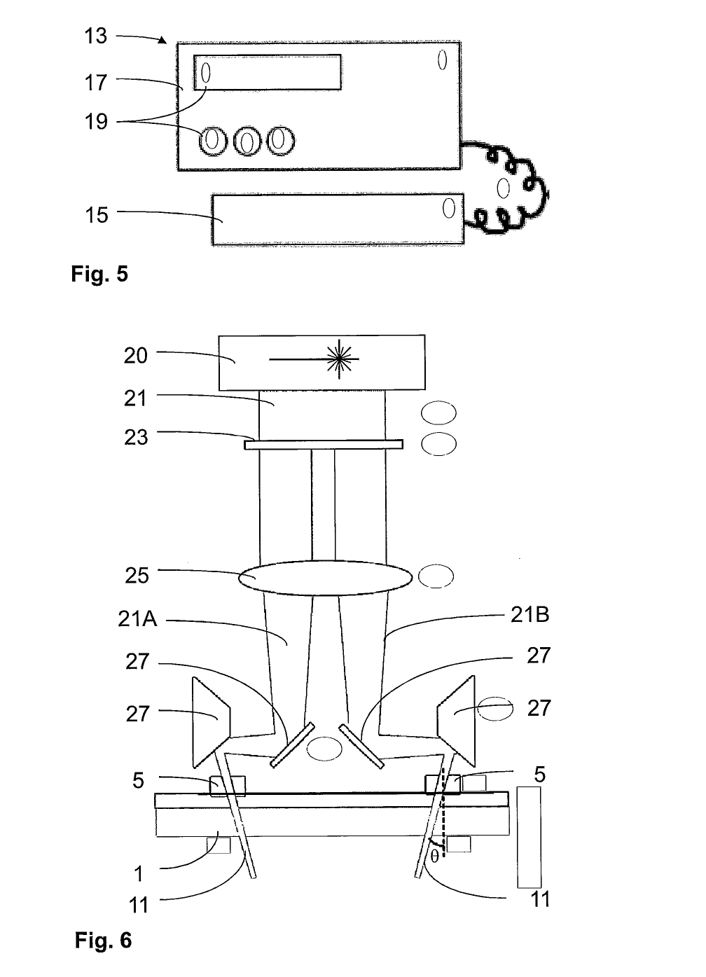

FIG. 5 shows a treatment system 13 comprising a treatment head 15 connected to a controller 17 comprising a user interface 19. The controller 17 may be wireless connected to the treatment head 15 and may be programmable, e.g. with a memory and/or by using an external data source such as a machine readable program storage medium. The treatment head 15 may be a handheld device. Here, the controller comprises a power source, e.g. a battery, but a separate power source, an electrical mains connection, etc. may be provided.

FIG. 6 shows a detail of a treatment head 15 for use in the treatment system of FIG. 5, comprising RF electrodes 5 in contact with a skin portion 1. The treatment head comprises a laser 20 providing a laser beam 21, which is controlled with suitable optics, here a beam splitter 23, a focusing system 25 and beam steering optics 27. Further optical elements like shutters, modulators, polarizers, filters etc may be provided as well. In FIG. 6, the laser beam 21 is split in a number of (here: two) beamlets 21A, 21B, which each are directed to illuminate the skin tissue and heat it to an elevated temperature to provide the first skin tissue portions with low inductance. Use of a single beam and/or plural laser is possible too, e.g. for heating plural skin tissue portions subsequently. The elevated temperature may be relatively low to provide transient heating. Preferably, the elevated temperature is relatively high, e.g. between about 60-80° C., such as the aforementioned 70° C., and/or the laser is used to ablate skin portions, so as to irritate the skin and invoke the rejuvenation process in assistance to the RF heating. Here, the beamlets 21A, 21B pass through the RF electrodes 5, providing an optimum overlap between the preheated skin tissue portions 11 and the electrodes 5 to improve coupling between the RF energy and the pre heated skin tissue portions 11. This may be realised by providing the RF electrodes 5 with a suitable aperture and/or by providing electrodes 5 with a conductive portion that is transparent to the laser radiation, e.g. Indium Tin Oxide (ITO) for near infrared radiation (e.g. up to about 1.5 micrometer wavelength) or Germanium for far infrared lasers (e.g. 10 micrometer wavelength).

It is noted that laser beam(s) need not be stationary and/or used for illumination at one position, but a laser beam position and/or angle may be adjusted with the appropriate optics, such as manually and/or machine adjustable optics e.g. piezo-mounted optics, acousto-optics, electro-optics, stepper motors etc, to provide different optical energy distributions and/or define plate-like heated or ablated shapes and/or more complex illumination profiles, concurrently and/or subsequently.

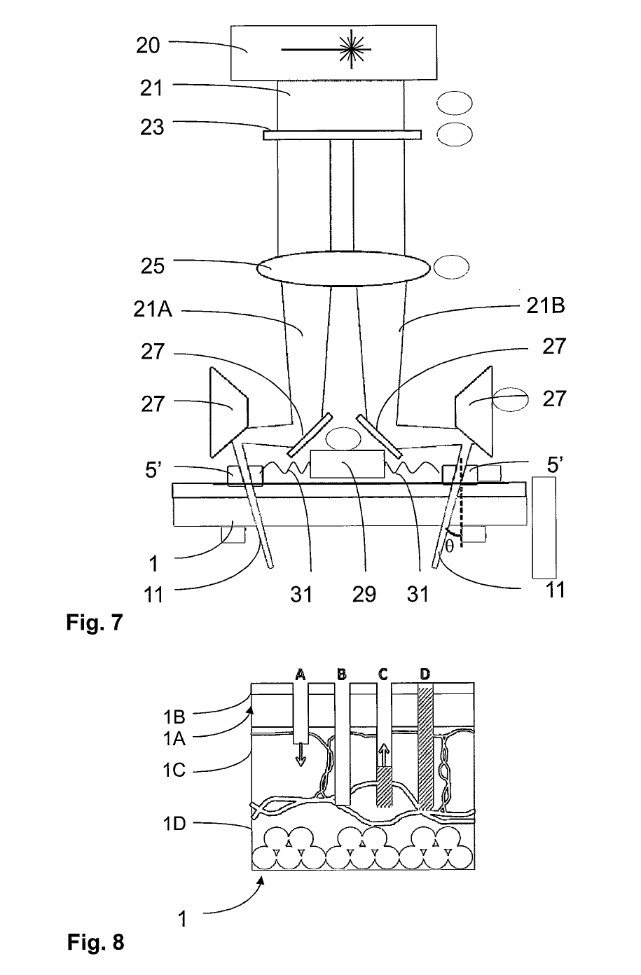

FIG. 7 shows a detail of a treatment head 15 similar to that of FIG. 6. Different, however, is that here the treatment head 15 comprises a dispenser 29 for a fluid, connected with fluid conduits 31 to RF electrodes 5′, which are configured to provide the fluid to the skin 1 at or near the interaction zone between the laser beam 21, the skin 1 and the RF electrodes 5. Instead of using specially formed electrodes, direct dispensing from the dispenser may also be used. The fluid may be a liquid, a gel, a cream etc, and may be used for improving electrical contact and/or impedance matching between the RF electrodes, soothing skin sensation, cooling or rather heating the skin, filling skin cavities, etc.

As set out above, it has been found out that ablating skin tissue producing one or more small cavities in the skin may invoke the rejuvenation process. FIG. 8 indicates that a cavity produced in the skin through the epidermis and dermis (FIG. 8 at A) and into the subcutis (FIG. 8 at B) may become fluid filled by the body with body fluids (FIG. 8 at C). The result is a highly-conductive portion in the skin extending for the length of the fluid-filled column which may extend up to the skin surface (FIG. 8 at D). More often than not, the body will continue producing fluid after the cavity has filled, providing a fluid layer on top of the skin tissue which allows a good electrical contact between a nearby RF electrode and the fluid-filled column. Such cavity may be produced by laser ablation, by perforating and/or by cutting with another technique. Laser cutting allows production of large numbers of very narrow cavities closely adjacent to each other, reducing discomfort to the treated subject yet providing a large-area (cross sectional area) for the first skin tissue portion 11. Another suitable method to produce a fluid-filled cavity is insertion of an injection needle into the skin tissue, and withdrawing the needle and fluid-filling of the cavity provided by the needle (not shown).

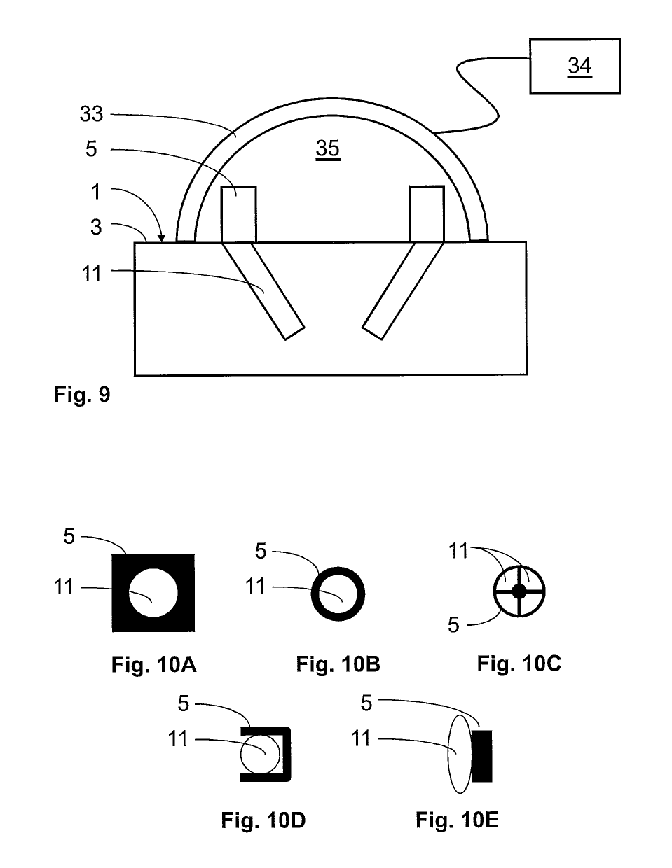

FIG. 9 shows a detail of another embodiment of a treatment head, comprising a vacuum dome 33 surrounding the RF electrodes and being connectable to a pump 34 providing a low-pressure volume 35 around the pre-treated skin tissue portions 11 with reduced pressure with respect to the outside atmosphere so as to suck body fluids into cavities produced in the skin tissue. A vacuum dome or other similar pressure difference device may be used as a stand-alone device possibly forming part of the treatment system, but need not be part of a treatment head. Also or alternatively a positive pressure may be applied around the cavity to force fluid into the cavity.

FIGS. 10A-10E show different geometries for RF electrodes 5 facilitating close contact between the low-impedance tissue portion 11 and the RF current, considered with respect to the shape of the low-impedance skin tissue portion 11 on the skin surface: rectangular or round electrode 5 surrounding the skin tissue portion (FIGS. 10A, 10B), a cross-hairstyle RF electrode with plural windows, a (rectangular) horseshoe electrode 5 or an elongated electrode 5 adjacent an elongated low-impedance skin tissue portion.

The basic principle of the method is that increasing the local conductance of the skin tissue enables RF energy to be guided to the treatment zone. This can be achieved by changing the tissue temperature or composition in the local zones to obtain a reduced impedance. Examples are simultaneous tissue heating or thermolysis of pre-defined geometries, e.g. pillars, plates, and/or combinations of these shapes leading to more complex zones, straight or angled zones, parallel edges or conical/tapered zones, etc. Subsequently, the RF energy will be applied via the thus prepared skin tissue zones.

The size, e.g. the diameter, of the photothermolysed or ablated tissue of first skin tissue portions is preferably about 1 micrometer or larger, preferably between 50-800 micrometer. Merely heated zones may be larger still. For laser-heated skin tissue, the water absorption coefficient of the tissue should preferably be >1 cm⁻¹. Light wavelengths in a range of about 0,1 micrometer to about 20 micrometer can be used to create the low-impedance skin tissue portions.

In skin treatment application systems relating to creation of photothermolysed lesions a focused pulsed laser at a wavelength of about 1 micrometer or longer may be used, preferably having a wavelength in the range of about 1.2-3 micrometer, with a pulse width of less than about 50 ms, preferably with a pulse length in the range of about 0.1-40 ms, with a fluence higher than about 1 J/cm², preferably between about 10-60 J/cm².

Suitable lasers and wavelengths for heating may be solid state lasers at a wavelength of about 1.3-1.5 micrometer, focused to produce heated skin tissue portions and/or lesions with typical dimensions of about 200-250 micrometer diameter or width, depending on the shape of the heated skin portion, although the diameter of the focus spot size may be smaller or larger. Suitable lasers may be pulsed at 9-11 mJ and 7.5-10 ms per pulse, resulting in a fluence of about 20-35 J/cm², and having a penetration depth into skin tissue of about 300 micrometer.

Suitable lasers and wavelengths for creation of ablative lesions may be solid state and/or gas lasers at a wavelength of about 2.5-11 micrometer. E.g. 2.9 micrometer wavelength Er:YAG, focused to about 100 micrometer diameter spot size, pulsed at 9-11 mJ and 2.5-5 ms per pulse. Or, CO₂ laser at 10.6 micrometer wavelength, focused to about 120-200 micrometer diameter spot size, at 50-80 mJ and 0.2-3 ms per pulse, and having a penetration depth of about 500-750 micrometer into human skin tissue. Pulsed CO lasers at a wavelength of 5.3 micrometer could also be used.

Skin ablation could also be provided by high power short pulse length lasers in the femtosecond range, e.g. Nd:YAG or Yb:YAG high power diode lasers. Further optical devices and techniques to provide suitable wavelengths, energies and/or heating or ablative effects may be suitably employed.

The treatment methods and systems disclosed herein may be used in a domestic environment but are also quite suitable for professional use for cosmetic treatment in a beauty salon, possibly in a cosmetic medical environment.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope. Elements and aspects discussed in relation with a particular embodiment may be suitably combined with elements and aspects of different embodiments within the scope of the appended claims.

1. A method of skin tissue treatment comprising the steps of:
determining a treatment zone within the skin tissue below the skin surface;
modifying an electrical conductance property of at least two first skin tissue portions present on opposite sides of the treatment zone with respect to a direction parallel to the skin surface;
after the step of modifying, providing radiofrequency energy to the treatment zone via said first skin tissue portions;
wherein the step of modifying said first skin tissue portions comprises decreasing electrical impedance for the radiofrequency energy, in particular increasing electrical conductance, of said first skin tissue portions relative to a second skin tissue portion present between said first skin tissue portions;
such that said first skin tissue portions extend into the skin tissue substantially from the skin surface to the treatment zone;
wherein the step of providing radiofrequency energy to the treatment zone comprises providing the radiofrequency energy with one or more radiofrequency electrodes in direct physical contact with one or more of said first skin tissue portions.

2. The method of claim 1, wherein said first skin tissue portions comprise a pair of elongated skin tissue portions having an elongated columnar or plate-like shape with a longitudinal axis, wherein the longitudinal axes of the elongated skin tissue portions of said pair converge toward each other in a direction from the skin surface toward the treatment zone.

3. The method of claim 1, wherein the step of modifying said first skin tissue portions comprises heating said first skin tissue portions.

4. The method of claim 3, wherein the step of modifying said first skin tissue portions comprises ablating skin tissue.

5. The method of claim 1, wherein the step of modifying said first skin tissue portions comprises providing one or more cavities in the skin tissue that are filled with an electrical conductive fluid.

6. The method of claim 5, comprising applying a pressure difference across one or more of said cavities between the skin tissue and the surrounding atmosphere for filling the cavity with body fluid.

7. (canceled)

8. A system for skin tissue treatment, in particular for performing the method of claim 1, comprising
a radiofrequency source for providing radiofrequency energy to a treatment zone within the skin tissue below the skin surface to heat said treatment zone, comprising a radiofrequency energy source with one or more radiofrequency electrodes;
a modifier configured to modify an electrical conductance property of at least two first skin tissue portions;
wherein the modifier is configured to decrease the electrical impedance for the radiofrequency energy, in particular increase electrical conductance, of said at least two first skin tissue portions relative to a second skin tissue portion present between said first skin tissue portions; and
wherein the one or more radiofrequency electrodes are configured for guiding the radiofrequency energy to the treatment zone through said first skin tissue portions when having said decreased electrical impedance;
wherein the first skin tissue portions are present on opposite sides of the treatment zone with respect to a direction parallel to the skin surface and extend into the skin tissue substantially from the skin surface toward the treatment zone;
wherein the radiofrequency electrodes are arranged for contact with the skin surface in a first pattern, and wherein the modifier is configured to provide said first skin tissue portions with respect to the skin surface in a second pattern; and
wherein the first and second patterns are substantially identical and wherein the radiofrequency electrodes are configured to be, in use, in direct physical contact with the first skin tissue portions.

9. The system of claim 8, wherein the modifier is configured to form a pair of said first skin tissue portions with a substantially elongated columnar or plate-like shape with a longitudinal axis and such that the longitudinal axes of the elongated skin tissue portions of said pair converge toward each other in a direction from the skin surface toward the treatment zone.

10. The system of claim 8, wherein the modifier comprises a laser configured to illuminate and heat the skin tissue for providing said first skin tissue portions.

11. The system of claim 10, wherein the laser is configured to ablate skin tissue.

12. The system of claim 8, wherein the modifier comprises a dispenser (29) configured to dispense one or more fluids in or on said first skin tissue portions (11).

13. The system of claim 8, wherein the modifier is configured to provide one or more cavities into the skin tissue and preferably comprises a pressurising device configured to apply a pressure difference across one or more of said cavities between the skin tissue and the surrounding atmosphere for filling the cavity with body fluid.

14. (canceled)

15. (canceled)
